(19) United States
(12) Patent Application Publication
(10) Pub. No.: US 2026/0102628 A1
(43) Pub. Date: Apr. 16, 2026

(54) METHOD AND APPARATUS FOR MONITORING OF PHOTODYNAMIC-THERAPY PRECURSOR STATES FOR ENHANCED THERAPEUTIC EFFICIENCY

(71) Applicants: Jerker WIDENGREN, Solna (SE); Joachim PIGUET, Skarpnäck (SE); Haichun LIU, Täby (SE); Elin SANDBERG, Stockholm (SE); Johan TORNMALM, Hägersten (SE)

(72) Inventors: Jerker WIDENGREN, Solna (SE); Joachim PIGUET, Skarpnäck (SE); Haichun LIU, Täby (SE); Elin SANDBERG, Stockholm (SE); Johan TORNMALM, Hägersten (SE)

(21) Appl. No.: 19/114,068

(22) PCT Filed: Sep. 22, 2023
(86) PCT No.: PCT/EP2023/076281

§ 371 Date: Mar. 21, 2025

(30) Foreign Application Priority Data
Sep. 23, 2022 (EP) 22197598.0

(51) Int. Cl. A61N 5/06; G16H 20/40

(52) U.S. Cl. CPC A61N 5/062; G16H 20/40; A61N 2005/0626; A61N 2005/0627; A61N 2005/063

(57) ABSTRACT

The invention regards a system comprising an excitation light source arranged to provide excitation light, and wherein the excitation light source is configured to illuminate a treatment location comprising a photosensitizer and wherein the excitation light source is configured to excite the photosensitizer into PDT precursor states; a sensing unit configured to monitor the fluorescence emitted by the excited photosensitizer; a processing unit configured for at least one iterative step of: —performing a pulse-train modulation of the monitoring excitation light characteristics in the time range of the PDT precursor state transitions of the photosensitizer, —obtaining PDT precursor state data by monitoring the fluorescence emitted by the photosensitizer, and —adapting the therapeutic excitation light and possible supply of PDT adjuvant compounds promoting PDT precursor state formation, based on the PDT precursor state data at and around the treatment location.

The present invention relates to spectroscopic methods, systems and compositions for excitation and monitoring of photodynamic state transitions in photosensitizer compounds for photodynamic therapy, at a location of treatment and surrounding regions.

### BACKGROUND

Photodynamic therapy (PDT) is a therapeutic approach with applications in areas including, but not limited to, ophthalmology, oncology, infectious diseases, dermatology and cosmetic treatments.

In PDT (FIG. 1), light (101) is irradiated onto a photosensitizer (PS), which upon excitation generates local cytotoxic effects. The mechanisms of PDT, as exerted by a PS upon light irradiation, are inherently complex. Traditionally, the mechanisms are divided into type I PDT and type II PDT. In both mechanisms, the PS is excited from a ground singlet state (102, S₀) to an excited singlet state (103, S₁), followed by intersystem crossing (104, ISC) into a non-emissive, dark triplet state (105, T₁). Type I (106) and type II (107) PDT mechanisms take place from the T₁ state (105). ISC denotes intersystem crossing.

In type I PDT, T₁ reacts directly with intracellular substrates and produces free radicals through hydrogen- or electron-transfer processes, which in turn can react with molecular oxygen (O₂) and water to generate harmful reactive oxygen species (ROS).

In type II PDT, the T₁ state of the PS is quenched by O₂, whereby the PS is brought back to the S₀ state (102), and O₂ is transformed into highly cytotoxic singlet oxygen (¹O₂).

For both type I and type II PDT the T₁ state (105) is thus a precursor state, with the PDT effect strongly coupled to the T₁ generation. One example out of many PS compounds, for which T₁ is the essential PDT precursor state is Methylene Blue (MB) as disclosed in Journal of Physical Chemistry A. 2011; 115(13):2702-7, “Effect of pH on Methylene Blue Transient States and Kinetics and Bacteria Photoinactivation”.

An example of a PS compound, for which \dot{R}⁻ is a prominent PDT precursor state is IRdye700DX (IR700). IR700 has recently emerged as an agent in photoimmunotherapy (PIT), in which IR700 is coupled to a monoclonal antibody targeting antigens expressed on cancer cells. Upon irradiation, IR700 can on the one hand generate toxic ROS levels via a type II mechanism, but can also generate cytotoxic effects via an uncaging mechanism, triggered by photoreduction into an \dot{R}⁻ state, as disclosed in Accounts of Chemical Research. 2019; 52(8):2332-9, “Near-Infrared Photoimmunotherapy of Cancer” and Nature Communications. 2016; 7:13378, “Near-infrared uncaging or photosensitizing dictated by oxygen tension”.

While PDT is a strongly emerging, non- or minimally invasive treatment, especially for various cancer diseases, several challenges remain for its further clinical use. The main challenges: i) finite tumor suppression, ii) poor tumor targeting, and iii) limited therapeutic depths are largely coupled to the PS used, which is also considered the most important element in PDT treatments, and are mainly attributed to the heterogeneity in disease biology and topography.

Given the decisive role of PS compounds in PDT treatments, much effort has been focused on the development of new PS compounds to overcome current challenges in PDT, for example to rationally modify PS compounds with photophysical properties adapted to specific tumor microenvironment (TME).

Generally, in the development of new, more potent and selective PDT regimes, it is beneficial to promote PDT precursor states of PS compounds, such as T₁, \dot{R}⁺ and \dot{R}⁻ states, strongly correlated to the PDT effect. At the same time, such states are strongly influenced by the local environment, and are often less promoted under TME conditions. The TME conditions can show large variations in e.g. local oxygen concentrations, redox conditions and pH, which in turn can depend on e.g. stage, type and localization of the tumor, and can be altered during the course of ongoing treatments. Therefore, it can be difficult to optimize the PDT effects of a PS by design only, so that it can maintain high and tumor-selective PDT effects over the broad spectrum of TMEs that can be encountered. Likewise, local oxygenation and redox conditions in and around e.g. a skin lesion to be treated by PDT can vary in time and space, and before, under, and after PDT treatment.

### SUMMARY

Thus there exists a need to optimize the PDT effects for different TME conditions, and treatment localizations in general.

The present inventors have realized that information of the PDT precursor state population of the PS, within and/or around the treatment location, can be used in order to optimize the PDT effects.

By adapting a monitoring excitation light modulation within the time range of the PDT precursor state transitions of the PS, information about the PDT precursor state populations in situ, at the treatment location(s) and/or other locations(s) can be obtained.

This can provide additional, orthogonal parameters for optimizing the PDT effects, beyond the optimization of the PS compound itself, or from knowledge of its mere concentration, thereby allowing for adaptation of the spatial distribution of a continuous wave (CW) or pulsed excitation within the treatment location.

Hence, with these PDT precursor states monitored in situ, at and around the treatment locations, a therapeutic excitation light can be adapted to maximize the PDT effects for different TME conditions, and treatment localizations in general.

The monitoring of the PDT precursor state populations builds upon the knowledge of transient state (TRAST) spectroscopy/imaging technique, as disclosed in Analytical Chemistry. 2007; 79(9):3330-41, “Monitoring kinetics of highly environment sensitive of fluorescent molecules by modulated excitation and time-averaged fluorescence recording”, and Journal of the Royal Society Interface. 2010; 7(49):1135-44, “Fluorescence-based transient state monitoring for biomolecular spectroscopy and imaging” which are hereby incorporated herein by reference in their entirety.

TRAST is a method, where a corresponding excitation modulation is used, in the time-range of the dark, photo-induced states of fluorophores to be followed. In TRAST, the population kinetics of dark, photo-induced states of fluorophores, such as T₁, \dot{R}⁺ and \dot{R}⁻, can be monitored via the time-averaged fluorescence intensity, <F>, or time-resolved fluorescence intensity, F(t), and from how <F> or F(t) changes due to population of dark states in the fluorophores upon systematic variation of the excitation modulation. By varying the excitation modulation over the same time range as the transitions of dark and/or weakly emitting states of the fluorophores, e.g. their T₁, \dot{R}⁺ and/or \dot{R}⁻ states, significant contrasts in the populations of these states (and in <F>) can be obtained, which can be used to determine their kinetics (FIG. 3).

Thus, the present disclosure relates, in a first aspect, to a system comprising:
an excitation light source arranged to provide pulse-train modulated monitoring excitation light and a pulse-train modulated or continuous wave therapeutic excitation light, and wherein the excitation light source is configured to illuminate a treatment location comprising a PS and wherein the excitation light source is configured to excite the PS into PDT precursor states;
a sensing unit configured to monitor the fluorescence emitted by the excited PS;
a processing unit configured for at least one iterative step of:
   performing a pulse-train modulation of the monitoring excitation light characteristics in the time range of the PDT precursor state transitions of the PS,
   obtaining PDT precursor state data by monitoring the fluorescence emitted by the PS, said PDT precursor state data indicative of the PDT precursor state population of the PS and/or its kinetics,
   adapting the therapeutic excitation light based on the PDT precursor state data at and around the treatment location, such that toxic effects of the PDT at the intended treatment location are enhanced, without correspondingly enhancing unwanted side-effects in locations surrounding the intended treatment location.

In this way, the basic principles of the TRAST technique can be applied to the monitoring and imaging of different dark state transitions, reflecting microenvironmental conditions and molecular interactions typically not detectable via other fluorescence parameters. The TRAST technique can be implemented in several different spectroscopic and microscopic modalities, and for studying a broad range of different samples. By the provision of such information, based on which PDT treatments are then further adapted and optimized to variations in time and space of local environmental conditions.

In a further aspect, the present disclosure relates to a method for PDT treatment, wherein at least one excitation light source illuminates a treatment location with excitation light, including pulse-train modulated monitoring excitation light and/or a pulse-train modulated or continuous wave therapeutic excitation light, wherein a PS absorbs said excitation light and thereby can undergo transitions into PDT precursor states, such as triplet (T₁), photo-reduced (\dot{R}⁻) and photo-oxidized (\dot{R}⁺) states, correlated to which states toxic effects can be generated, such as formation of free radicals, reactive oxygen species (ROS), and singlet oxygen, or cell membrane damage, or other cytotoxic effects, and where the procedure further includes at least one iterative step of:
i. performing a pulse-train modulation of the monitoring excitation light characteristics in the time range of the PDT precursor state transitions of the PS,
ii. obtaining PDT precursor state data by monitoring the fluorescence emitted by the PS, said PDT precursor state data indicative of the PDT precursor state kinetics and population of the PS,
iii. adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

A PDT adjuvant as used herein refers to a compound which is configured to, directly and/or indirectly, promote formations and/or populations of PDT precursor states in a PS used for PDT. This can take place via mechanisms including enhanced intersystem crossing (ISC) to a T₁ state, enhanced photo-reduction to \dot{R}⁻ and enhanced photo-oxidation to \dot{R}⁺, as further disclosed below.

In a further aspect, the present disclosure relates to a computer-implemented method comprising a method for PDT treatment, as disclosed elsewhere herein.

In a further aspect, the present disclosure relates to a computer program product comprising instructions which, when the program is executed by a computer, cause the computer to carry out the steps of a method for PDT treatment, as disclosed elsewhere herein.

In a further aspect, the present disclosure relates to an apparatus, such as a computer, comprising a memory comprising the computer program as disclosed elsewhere herein.

In a further aspect, the present disclosure relates to a composition comprising a PS and a PDT adjuvant for use in medical treatment.

In yet a further aspect, the present disclosure relates to a PS for use in a method of PDT treatment of cancer, wherein the method is as disclosed elsewhere herein.

### DESCRIPTION OF THE DRAWINGS

In the following, embodiments and examples will be described in greater detail with reference to the accompanying drawings:

FIG. 1 shows a schematic illustration of an example embodiment PS within a 3-state model,

FIG. 2 shows a schematic illustration of an example embodiment 4-state electronic state model of a PS compound, or fluorophore,

FIG. 3 shows a schematic illustration of transient state (TRAST) spectroscopy/imaging according to an embodiment of the present disclosure,

FIG. 4 shows a schematic illustration of interstitial PDT (IPDT) with optical fibers coupled to a fiber switch for guiding excitation light from one or several lasers to the region of PDT treatment according to an embodiment of the present disclosure,

FIG. 5 shows TRAST imaging implemented with wide-field microscopy and confocal laser scanning microscopy (CLSM) according to an embodiment of the present disclosure,

FIG. 6 shows a schematic illustration of a system for optical fiber-coupled TRAST measurements according to an embodiment of the present disclosure, and experimental results obtained by said system.

### DETAILED DESCRIPTION

Microenvironmental differences (for example tumour microenvironmental differences) as used herein refers to differences in local environmental conditions that influences PS compounds, the PDT precursor state populations and/or kinetics, as is known from the literature. Examples of Microenvironmental differences include (local) oxygen concentration, redox condition and pH.

Toxic effects as used herein refers to toxic effects originating from PDT precursor states of PS compounds in PDT. Such toxic effects are known from the literature, and includes for example formation of free radicals, reactive oxygen species (ROS), and singlet oxygen, or cell membrane damage.

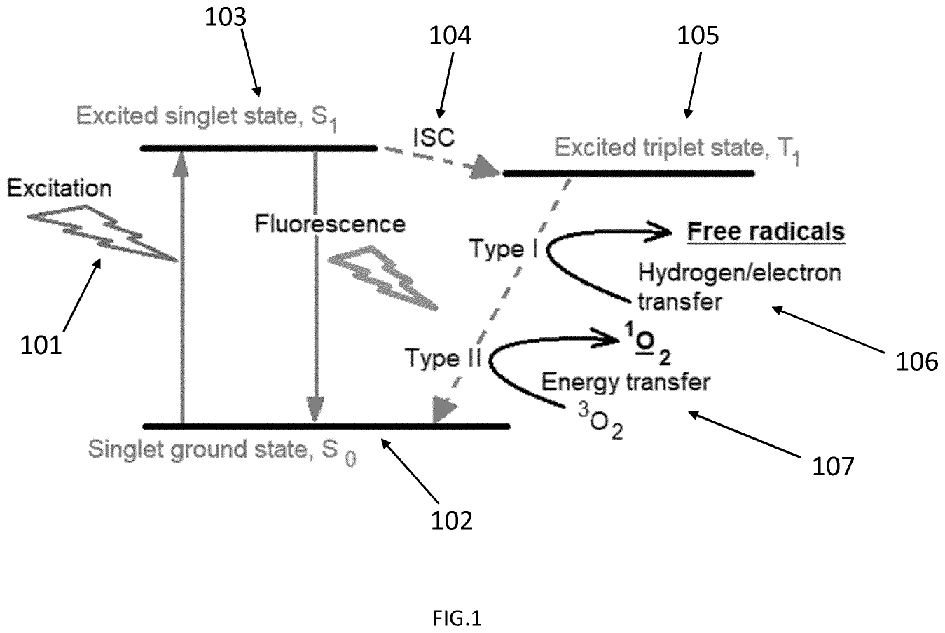

FIG. 1 shows, as described above, a schematic view of a PS within a 3-state model, comprising the singlet states S₀ and S₁, undergoing multiple excitation-emission cycles, and a non-emissive, dark triplet state, T₁. Type I and type II PDT mechanisms take place from the T₁ state. ISC denotes intersystem crossing

However, also other photoinduced states of the PS, such as photo-induced radical states (\dot{R}⁺ and \dot{R}⁻), can be implicated in the PDT mechanism, and with the PDT effect then strongly coupled to the population of such states as further disclosed in Nature Communications. 2016; 7, “Near-infrared uncaging or photosensitizing dictated by oxygen tension”.

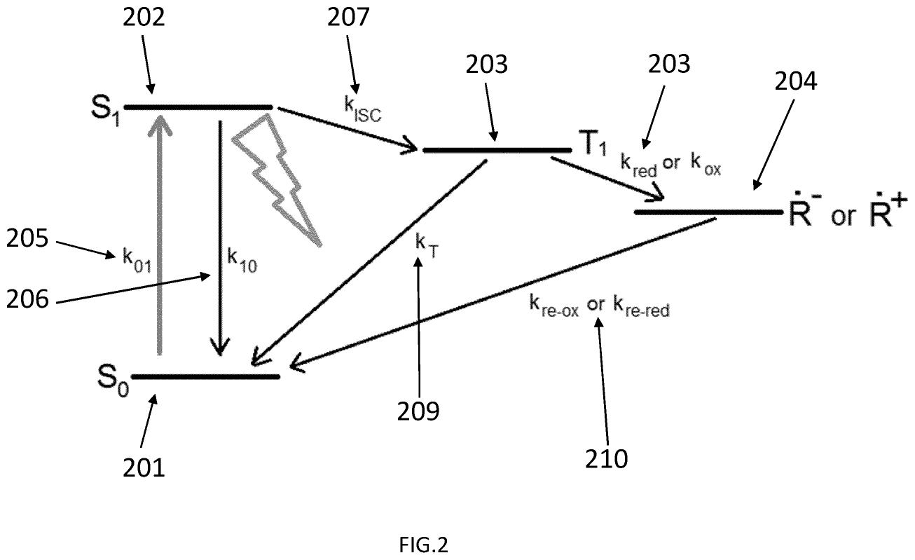

With photo-induced radical states included, the PS photodynamics can in a simplified scenario be modeled by a 4-state model (FIG. 2).

Upon excitation, the PS compound can transit back and forth between an emissive form (the singlet states S₀, 201, and S₁, 202, undergoing multiple excitation-emission cycles, taking place typically in the nanosecond time range) and non-emissive, dark states (e.g. triplet, T₁, 203, and photo-ionized radical states, \dot{R}⁻/\dot{R}⁺, 204). The transitions to and from T₁ typically takes place on a microsecond-to-millisecond time range, while the transitions to and from \dot{R}⁻/\dot{R}⁺ typically take place on a slower sub-millisecond-to-millisecond time range. Both these transitions are highly environment sensitive, in particular to local oxygen concentrations and redox conditions. k₀₁ (205) is the excitation rate, k₁₀ (206) the S₁ (202) decay rate, k_{ISC} (207) the intersystem crossing rate from S₁ to T₁, k_{T} (208) the decay rate of T₁ to S₀. k_{red} (209) and kₒₓ(209) denote reduction and oxidation rates from T₁ leading to formation of \dot{R}⁻ and \dot{R}⁺, respectively, and kᵣₑ₋ₒₓ (210) and k_{re-red} (210) are the recovery rates of \dot{R}⁻ and \dot{R}⁺, respectively, back to S₀. Light-induced generation of \dot{R}⁻ and \dot{R}⁺ can occur differently for different fluorophores and PS compounds, e.g. also from S₁ or from higher excited singlet and triplet states as disclosed in Journal of Physical Chemistry A. 2007; 111(3):429-40 “Strategies to improve photostabilities in ultrasensitive fluorescence spectroscopy” and Anal Chem. 1998; 70(13):2651-9 “Photobleaching of fluorescent dyes under conditions used for single-molecule detection”.

As described above, main challenges of PDT include; i) finite tumor suppression, ii) poor tumor targeting, and iii) limited therapeutic depths. To tackle these challenges, prior art PDT development has mainly focused on PS optimization by molecular design.

However, PDT precursor states of PS compounds, such as T₁, \dot{R}⁺ and \dot{R}⁻ states, are largely influenced by the local environment, and are often less promoted under e.g. tumor microenvironment (TME) conditions. TME conditions can show large variations, in e.g. local oxygen concentrations, redox conditions and pH, depending on e.g. stage, type and localization of a tumor, and can be altered during the course of ongoing treatments. Similarly, local oxygenation and redox conditions in and around e.g. a skin lesion to be treated by PDT can vary in time and space, and also before, under, and after PDT treatment. In general, it is difficult to optimize PDT effects and address the above challenges i), ii) and iii) by PS design only.

In view of this, there is a strong need for systems and associated methods for obtaining important, additional, orthogonal feedback, in the form of in situ mapping of the PDT precursor state populations of the PS compound.

The present disclosure can provide such key information, based on which PDT treatments can then be adapted and optimized to variations in for example time and space of local environmental conditions.

In a first aspect, the present disclosure relates to a system that is configured to provide an excitation modulation in the time range of PDT precursor state transitions of PS compounds as a means to provide this key information, adding flexibility, selectivity and efficiency to PDT sessions.

By excitation modulation in the time range of the PDT precursor state transitions of PS compounds, and monitoring the response in the fluorescence emission from the PS compounds at the localizations of the PDT treatment, data of these precursor states, before, during and after PDT can be made possible.

Based on this feedback, providing an additional orthogonal adjustment variable, the excitation, i.e. the therapeutic excitation light, can then be adapted to regulate the PDT effects for example for different TME conditions, and at different PDT treatment localizations in general.

Moreover, this feedback can also be used in order to guide the supply of PDT adjuvant compounds, directly or indirectly affecting PDT precursor state transitions at the treatment location, thereby providing an additional means to adjust and optimize PDT effects. I

PDT precursor state data, such as the state populations, the photochemical rates, and how they are influenced by the excitation light applied locally, and/or any supply of PDT adjuvant compounds, are unavailable for many common PS compounds. In particular, there is a great potential for determining in vivo photochemical rate parameters for use in PDT modeling and dosimetry (Kim M et al, Phys. Med. Biol. 62 (2017) R1-R48).

The present disclosure can provide such information, by monitoring photo-induced PDT precursor states by their non- or close to non-existing emission, via the correlated changes in the relatively strong fluorescence emission from the parent PS (typically excited singlet) state. This is different from detection of the emission from the photoproducts themselves, as previously suggested (PCT WO 99/06113). This emission is much weaker than that of the parent PS states, and to distinguish this emission, a distinct spectral shift of this emission would also be required.

The presently disclosed embodiment can be compatible with existing PDT regimes, and offers additional functionalities, flexibility and optimization variables to such regimes.

Thus, the present disclosure relates to a system comprising one or more of an excitation light source, a sensing unit and/or a processing unit.

Typically, the excitation light source is arranged to provide pulse-train modulated monitoring excitation light and a pulse-train modulated or continuous wave therapeutic excitation light. The light source may comprise one or multiple light emitters, for example one light emitter of the monitoring excitation light and one light emitter of the therapeutic excitation light. In any event, the excitation light source is typically arranged so that it independently can provide the monitoring excitation light and the therapeutic excitation light. Typically, the monitoring excitation light is a pulsed light (pulse-modulated light). Typically, the therapeutic excitation light is either a pulse-modulated light or a continuous wave light.

Typically, the excitation light source is configured to illuminate one or more treatment locations and/or surrounding locations comprising a PS. Typically the monitoring excitation light and/or the therapeutic excitation light is arranged to excite the PS into PDT precursor states.

Typically, the sensing unit is configured to monitor fluorescence, preferably fluorescence emitted by the excited PS. For example, wherein the PS is present at the one or more treatment locations and/or the one or more other locations.

Preferably, the processing unit is configured for carrying out one or more iterations of one or more method steps. Typically said method steps include at least a step of performing a pulse-train modulation of the excitation light (e.g. the monitoring excitation light). Said pulse-train modulation is preferably arranged such that it is carried out in the time range of the PDT precursor state transitions of the PS.

Preferably, said method further includes a step of obtaining PDT precursor state data, such as at the treatment location, such as by monitoring the fluorescence emitted by the PS. Thus, the step of obtaining PDT precursor state data may be referred to as a step of monitoring PDT precursor state data and/or as a step of measuring PDT precursor state data and/or as a step of determining PDT precursor state data. Said PDT precursor state data may include for example PDT precursor states and the photochemical rate parameters of the PS compounds used. Said PS may preferably be located at the one or more treatment location, said PDT precursor state data is typically indicative of the PDT precursor state population of the PS and/or its kinetics.

The photodynamics of PDT precursor states of a PS, such as T₁, \dot{R}⁺ and \dot{R}⁻ states, are preferably monitored on the time scales of their transitions, typically on a micro- and millisecond time scale, and at potential locations for PDT therapy. This monitoring may thus provide information on how to optimally design the distribution of excitation light in time and space to optimize the PDT precursor state populations and the PDT effects in relation to the light dose.

Preferably, the method further includes a step of adapting the therapeutic excitation light. Said adaptation is typically made based on the PDT precursor state data obtained at and around the treatment location. The adaptation is typically carried out such that toxic effects of the PDT at the intended treatment location are enhanced, without correspondingly enhancing unwanted side-effects in locations surrounding the intended treatment location.

Thus, while optimization of the PDT treatment may comprise providing one or more PDT adjuvants to a treatment location, optimization of PDT treatment may alternatively, or additionally, include the adaptation of the therapeutic excitation light. For example, the excitation light may be regulated in time and space to optimize the localization (spatial specificity) and efficiency of the treatment, based on the monitoring of the local PDT precursor states. The adaptation of the therapeutic light can be done with the same and/or additional light (e.g. laser) sources, as well as the same and/or additional light emitters of the same light source as used in the monitoring step, i.e. the monitoring excitation light. Additionally, or alternatively, the PDT treatment may be optimized by locally providing one or more PDT adjuvant compounds, preferably wherein said provision is adapted based on feedback from the monitoring step.

The monitoring of the PDT precursor state population in a PS can also be performed globally in a tomographic approach, such as on top of the Fluorescence Diffuse Optical Tomography (FDOT) procedure.

Tomographic systems to image sub-surface PS distribution have also been developed based on fluorescence measurements of the PS, Opt. Lett. 2009; 34(3):232-4 “In vivo photosensitizer tomography inside the human prostate” and J. Biomed. Opt. 2013:18(4):046008 “White light-informed optical properties improve ultrasound-guided fluorescence tomography of photoactive protoporphyrin IX”. In such FDOT approaches, a series of fluorescence measurements are performed with different light-source-detector pairs, using different fiber pairs. Based on the fluorescence intensities detected for different source-detector fiber pairs, an estimate of the PS distribution, η(r), is obtained by minimizing the residual between forward model calculated fluorescence intensities (with proper normalization) for η(r) and the measured ones. This minimization problem is ill-posed, and thus regularization is used to single out a useful and stable solution. When employing Tikhonov regularization, the regularized solution can be found by minimizing the quantity

$${χ}^{2} = {‖ δ ( η ) ‖}^{2} + λ {‖ L [ η - {η}_{0} ] ‖}^{2} . \qquad ( 1 )$$

Here δ(η)=Γ−F(η), where F(η) is the forward model calculated fluorescence intensity, F the measured fluorescence intensity, A is the regularization parameter, L is a regularization matrix, and η₀ is the initial estimate of the PS distribution. Discretizing the geometry into elements, defined by voxels, the forward model is described by

$$F ( η ) = \frac{1}{{U}_{x} ( {r}_{s} , {r}_{d} )} {\sum }_{n = 1}^{NN} {CU}_{m}^{*} ( {r}_{d} , {r}_{n} ) {U}_{x} ( {r}_{s} , {r}_{n} ) Δ V {η}_{n} , \qquad ( 2 )$$

where rₛ, r_{d} and rₙ denote the coordinates for the light source, detector, and internal voxel, respectively. Uₓ and Uₘ represent the forward solution of the excitation light and fluorescence light, respectively, while

$${U}_{x}^{*} and {U}_{m}^{*}$$

are the adjoint solutions to the forward fluorescence problem. NN is the total number of voxels, ΔV is the voxel volume, C is a constant depicting the fluorescence efficiency of the PS, ηₙ is the PS concentration for voxel n. This inverse problem can be solved using an iterative scheme to reconstruct η(r). Denoting the parameter estimate for iteration i as ηᵢ, to find the parameter ηᵢ that minimizes equation (1), the first-order derivative of χ² is set to zero. i.e., ∂χ²/∂η=0. In matrix notation this leads to (16)

$$\frac{\partial {χ}^{2}}{\partial η} = {J}^{T} δ ( {η}_{i} ) - λ {L}^{T} L [ {η}_{i} - {η}_{0} ] = 0 , \qquad ( 3 )$$

where J denotes the Jacobian defined by J=∂F/∂η. Using a Taylor series to linearize the forward model in equation (2), i.e., F(ηᵢ)≈F(ηᵢ₋₁)+J[ηᵢ−ηᵢ₋₁], the parameter estimate can be updated using

$${η}_{i} = {η}_{i - 1} + {( {J}^{T} J + λ {L}^{T} L )}^{- 1} \times [ {J}^{T} δ ( {η}_{i - 1} ) - λ {L}^{T} L {η}_{i - 1} ] , \qquad ( 4 )$$

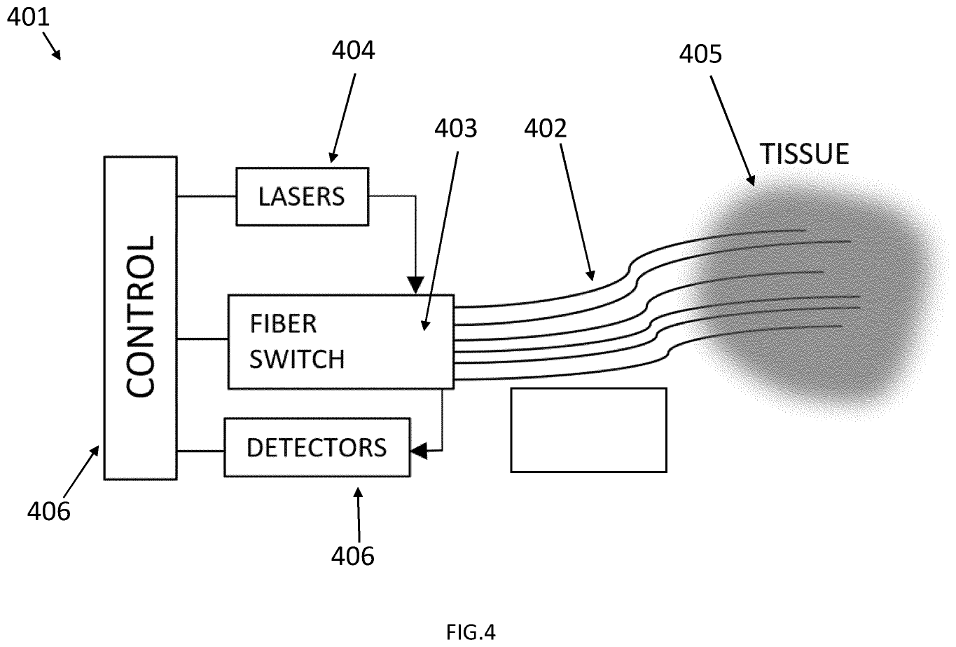

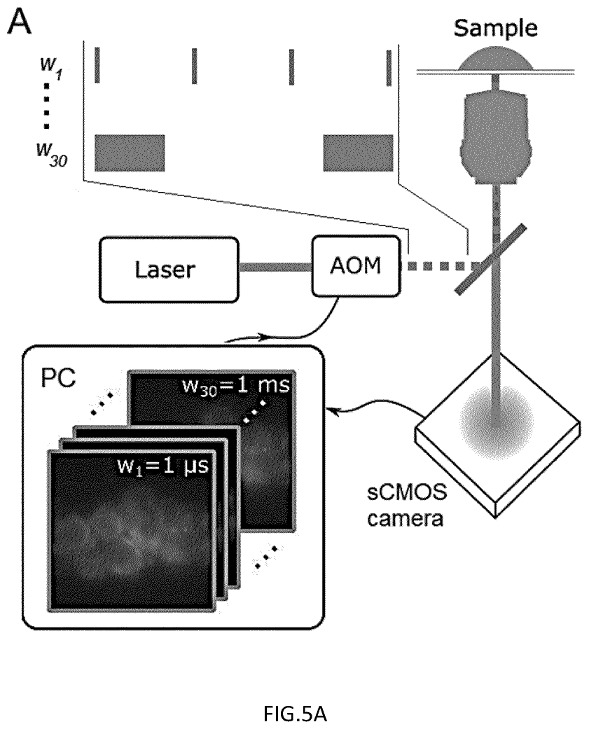

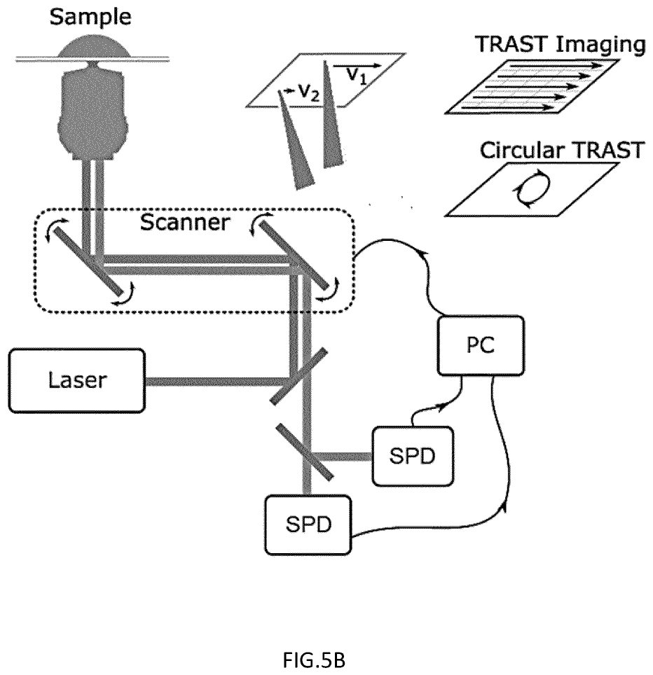

which will eventually reconstruct the spatial distribution of the PS in combination with some stopping criteria. This may be experimentally implemented using a multitude of optical fibers as in IPDT (FIG. 4).

The PDT precursor state population of a PS can also be reconstructed in a similar manner. This may be achieved through the following two major steps:
X) FDOT determination of PS spatial distribution η(r), employing CW excitation and fluorescence intensity measurements. This can be done by solving the minimization problems e.g. the one specified in equation (1), then using Tikhonov regularization and a certain forward model, e.g. equation (2). η(r) can be reconstructed in this step.
Y) Integration of the TRAST approach with the FDOT measurements, employing pulse-train modulation of the excitation light characteristics in the time range of the PDT precursor state transitions of a PS for PDT treatment and measuring the pulse-duration-dependent time-averaged fluorescence intensities, [symbol] F(η,w) [symbol] , where [symbol] F(η,w) [symbol] can then be modeled by

$$〈 F ( η , w ) 〉 = \frac{1}{{U}_{x} ( {r}_{s} , {r}_{d} )} {\sum }_{n = 1}^{NN} {CU}_{m}^{*} ( {r}_{d} , {r}_{n} ) {U}_{x} ( {r}_{s} , {r}_{n} ) {TF}_{n} ( w , {k}_{01} , {k}_{10} , {k}_{T} , {k}_{ox / red} , {k}_{re - red / re - ox} ) Δ V {η}_{n} . \qquad ( 5 )$$

Here, the same notations are used as in equation (2), and TFₙ is the voxel-dependent transfer function that projects the influence of pulse width w and other rate parameters (voxel-dependent) into change in the time-averaged fluorescence intensity [symbol] F(η, w) [symbol] .

It can be noted that some simplifying assumptions can be made here; given linear PS compounds, the transfer function TFₙ is parameterized and can be described by an analytical solution when w is sufficiently small. Further, k₀₁ is linearly dependent on the excitation intensity by a factor of the excitation cross-section of the PS. Thus, with proper pre-characterization, k₀₁ is not a free variable. In addition, the decay rate of S₁, k₁₀, may be approximated as a constant. So the dimensions of the inner space of TFₙ can be much reduced.

With this knowledge of the TF and with predetermined η(r) by step X as an input, the rates characterizing the PDT precursor states for each voxel can be reconstructed using a similar iterative approach as in the CW FDOT problem (discussed above).

For the detection/imaging part, one or several lasers/LEDs providing additional modulated excitation in the time range of the PDT precursor state transitions of the PS used, can be fed into one or several optical fibers, which at the same time can be used for directing the excitation light, as described above.

Thus, the presently disclosed system preferably comprises a processing unit configured for at least one iterative step of:
performing a pulse-train modulation of the monitoring excitation light characteristics in the time range of the PDT precursor state transitions of the PS,
obtaining PDT precursor state data by monitoring the fluorescence emitted by the PS, said PDT precursor state data indicative of the PDT precursor state population of the PS and its kinetics,
adapting the therapeutic excitation light based on the PDT precursor state data at and around the treatment location, such that toxic effects of the PDT at the intended treatment location are enhanced, without correspondingly enhancing unwanted side-effects in locations surrounding the intended treatment location.

It should be noted that the step of obtaining may be performed one or more times together with the step of performing a pulse-train modulation, for example at least one time. In other examples, said steps are performed at least two times, such as at least three times, or at least five times. Additionally, the step of adapting may be performed before, in-parallel or after the steps of performing and obtaining. Thus the respective order of the steps is not necessarily important. However, in certain embodiments said steps are performed in the order of: performing, obtaining and adapting, either strictly subsequently or wherein at least two steps partly or completely overlap,

Thus, the build-up of PDT precursor states in a PS, subject to a certain excitation modulation (the step of performing a pulse-train modulation), can be quite different at the location of a lesion to be treated, compared to at a location close to this lesion, or at the location of the lesion over time.

Monitoring the PDT local precursor state population(s) of a PS, such as its photochemical rates, at and around the location of a lesion, as part of a PDT treatment procedure (the step of obtaining), typically has three major purposes: First, it can add spatial selectivity to the PDT treatment. Based on feedback from this monitoring, the treatment excitation (the step of adapting) can be adapted to optimize PDT precursor state populations of the PS at the location of the lesion and minimize the same populations in surrounding regions, where the local environmental conditions (and thus likely also the PDT precursor state kinetics) are different than at the lesion. Second, it makes it possible to iteratively monitor the PDT precursor state build-up over time (by the steps of obtaining and of performing a pulse-train modulation) and adapt the excitation light (the step of adapting) to for example maintain high populations of the precursor state(s) at the lesion, during the course of the PDT treatment, thereby enhancing the effect of this treatment. Third, it provides feedback on the effects of possibly added PDT adjuvant compounds, on the PDT precursor state populations and kinetics, at and around the treatment location. Thereby, the spatial and temporal supply of PDT adjuvants can be likewise adapted for enhancing the treatment effect(s).

Monitoring (e.g. TRAST monitoring) can be made compatible with a range of optical imaging/spectroscopy modalities for excitation time-modulation, combined with average fluorescence intensity detection. Such modalities include time-modulated wide-field excitation, moving arrays of laser foci or laser excitation fringe patterns, as well as various spatial confinement strategies of the excitation and/or the detection. Apart from realizing TRAST in a stationary confocal microscope arrangement, and TRAST implemented with wide-field microscopy (FIG. 5A), other experimental setups demonstrated for TRAST include total-internal-reflection microscopy with evanescent-field excitation, light-sheet excitation microscopy, and two-photon-excitation microscopy. Evidently, the time-modulated excitation experienced by a stationary sample can also be generated by translation of the sample with respect to the excitation, or vice versa. Based on this concept, TRAST imaging can also be well implemented in a laser scanning confocal microscope (LSCM) (FIG. 5B).

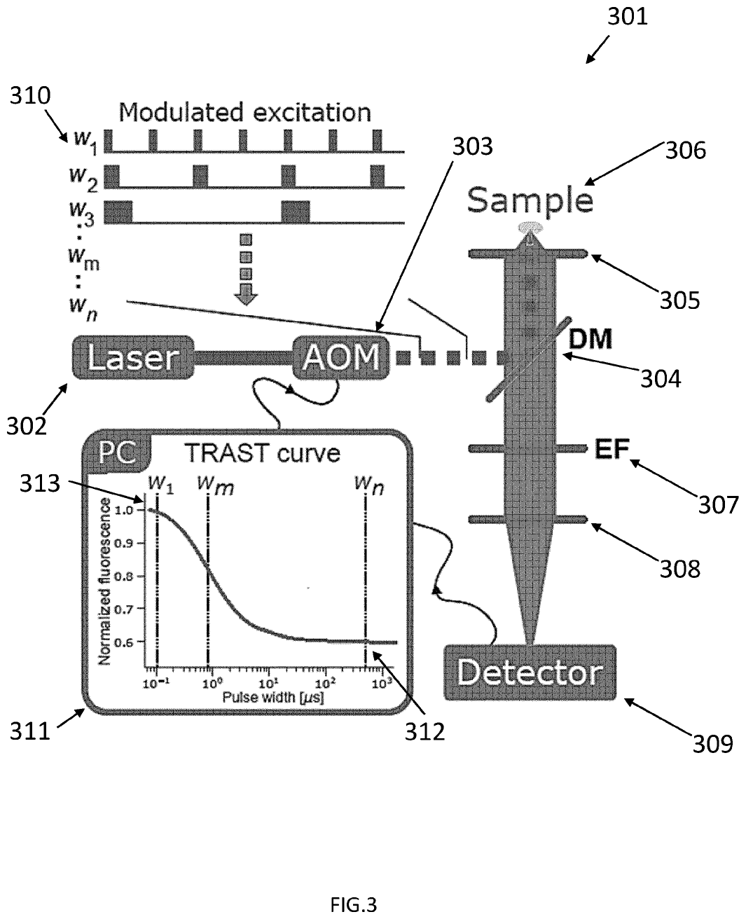

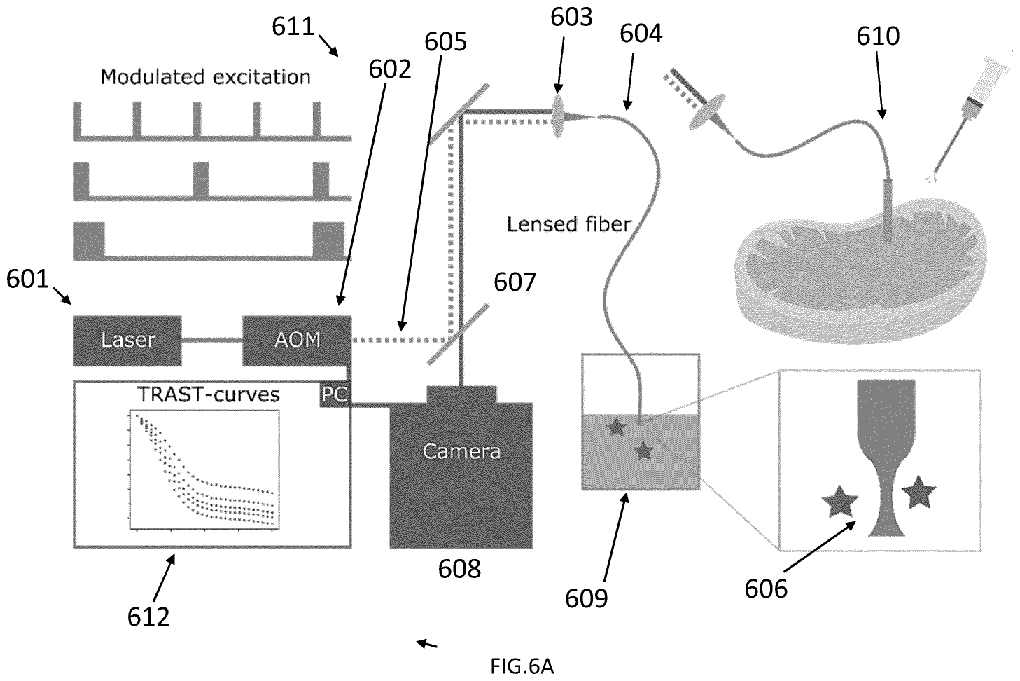

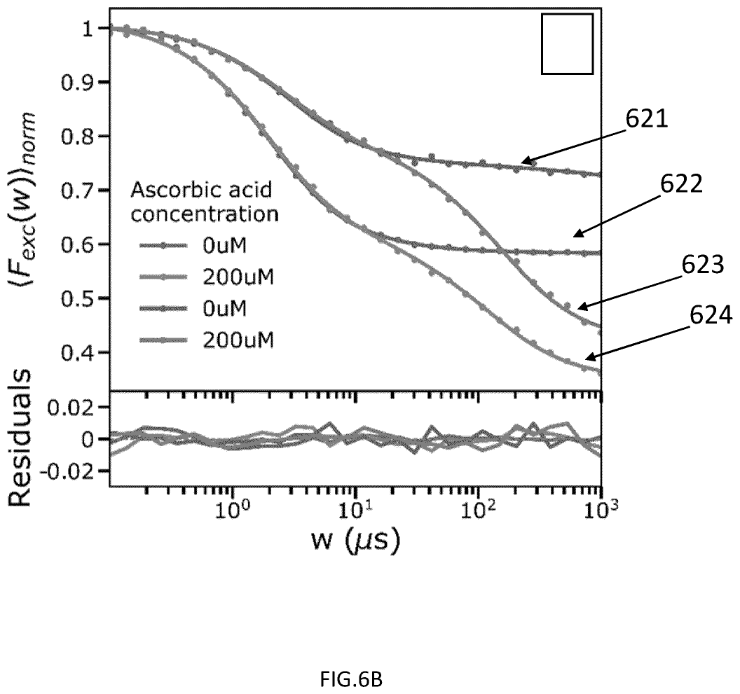

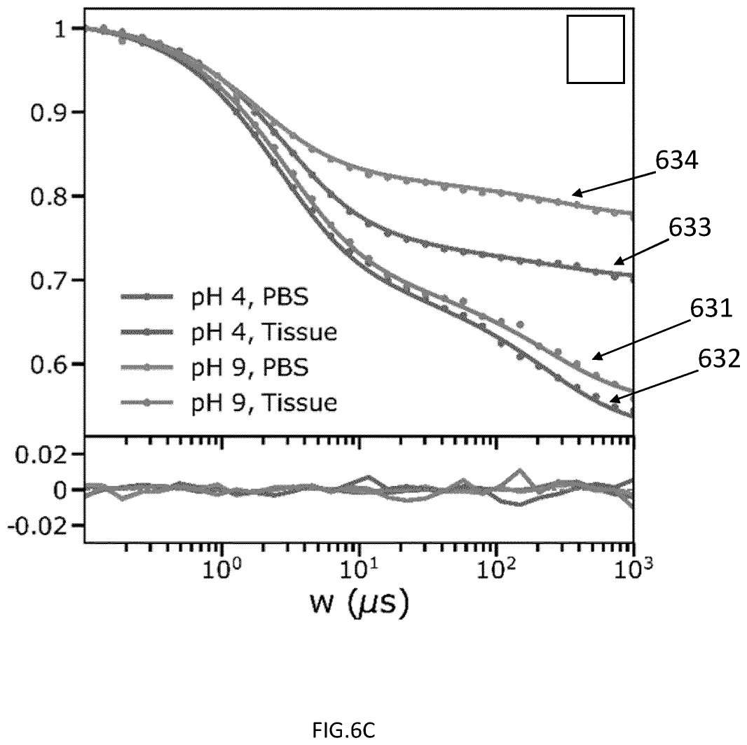

Monitoring of the PDT precursor state population in a PS (by the steps of obtaining and of performing a pulse-train modulation), for example by a TRAST approach (FIGS. 3 and 5), can be performed locally at the location(s) of individual fiber end(s), using the same excitation-light-delivering fiber(s) also as detection fiber(s) (FIG. 6A).

FIG. 6A illustrates the presently disclosed embodiment of a system for PDT (611), wherein the system is arranged such that the same excitation-light-delivering fiber(s) also are arranged as detection fiber(s). The system may thus be arranged to carry out a method for PDT as disclosed elsewhere herein.

The system (611) typically comprises an intensity-modulated excitation light (605), e.g. from a laser (601) and an AOM (602). The excitation light (605) is applied, e.g. lensed by an objective (603), via a gradient-index multimode optical fiber (604), into a sample (609, tissue or water solution). At the fiber end (606) in the sample, the modulated excitation light can generate prominent differences in the PDT precursor states of the PS compounds (of T₁ in MB, and of T₁ and \dot{R}⁻ in IR700), depending on the excitation pulse train characteristics applied (in this case by variation of w), as seen in the TRAST curves. The fluorescence plotted in the TRAST curves (612), corresponds to the (average) fluorescence from the PS compounds generated by the (modulated) excitation at the fiber end, which was collected (606) by the same fiber (604), passed through the fiber, through a dichroic mirror (607), and was then detected in a region-of-interest (ROI) within the field of detection of a camera (608). While only one fiber is shown in FIG. 6A, the system may comprise multiple fibers, into which excitation light from one or several lasers is introduced, and via which fluorescence detected from their fiber-ends is fed to different ROIs detected on the camera pixel array, and/or to a set of point detectors. Further, the fiber(s) may for example either be kept in solution (609) with dissolved fluorophores/PS (e.g. MB or IR700) or be placed in a piece of tissue through a cannula (610).

FIG. 6B shows TRAST curves recorded from IR700 by the setup shown in FIG. 6A, recorded of tissue with ascorbic acid (623), of tissue without ascorbic acid (621), as well as of PBS buffer solution with ascorbic acid (624) and of PBS buffer solution without ascorbic acid (622).

As can be seen in the experimental results, addition of ascorbic acid led to a prominent relaxation in the time range of 0.1 ms. This can be attributed to the build-up of a \dot{R}⁻ state population in IR700, a known PDT precursor state of IR700. Ascorbic acid may thus serve as a possible PDT adjuvant. A faster major relaxation in the TRAST curves is also seen in the microsecond time range, and is attributed to T₁ build-up, another major PDT precursor state.

FIG. 6C shows TRAST curves, corresponding to those of FIG. 6B, recorded of MB diluted in buffer solutions at different pH, and recorded with the fiber-end placed in tissue at pH 4 (632), in tissue at pH 9 (631), in PBS buffer solution at pH 4 (633) and in PBS buffer solution at pH 9 634).

For monitoring of PDT precursor state populations, the excitation is preferably modulated in the time range of the PDT precursor state transitions of the PS. For most PS compounds, and similar to most organic fluorophores, transitions between an emissive state (S₁) and T₁ typically takes place on a microsecond-to-millisecond time scale (see examples of MB and IR700 in FIGS. 6B and 6C, respectively).

Transitions to and from \dot{R}⁺ and \dot{R}⁻ states, of organic fluorophores as well as of PS compounds (FIGS. 6B and 6C), typically take place on a longer time scale, sub-ms-to-ms. As practiced in TRAST experiments (FIG. 3), large differences in the population of a state (T₁, or \dot{R}⁺ and \dot{R}⁻ states) can be generated by applying excitation with pulses shorter than the time range of the transitions to and from this state (or of the relaxation time of the population build-up of this state), compared to excitation pulses with pulse durations longer than this time range/relaxation time.

The excitation intensity onto the sample, e.g. treatment location, must typically have a certain intensity. In general the intensity should be at least such that PDT steady-state populations of precursor states of a PS, such as T₁, \dot{R}⁺ and \dot{R}⁻ states, can be generated in the PS compound entities when the excitation is active.

However, given for example the strong ISC yields of most PS compounds used for PDT (and that additional PDT precursor states than T₁ are often generated following T₁ generation) the irradiances needed onto the monitored/targeted region, Φ_{exc}, can generally be much lower than in typical TRAST experiments.

Moreover, applying pulsed excitation with lower excitation duty cycles (˜1%), Φ_{exc} can be high within the time of the excitation pulses, but the average Φ_{exc} experienced by a sample over the time of the whole pulse train can still be kept within allowed levels for PDT, and so can the total excitation light doses Φ_{exc} integrated over the duration of the pulse train(s) applied). When multiple light sources are used, appropriate Φ_{exc} distributions can be ascertained by using e.g. a Cimmino algorithm to control the powers and positions of the excitation light sources, as described in Med. Phys. 2005; 32(12):3524-36, “Optimized interstitial PDT prostate treatment planning with the Cimmino feasibility algorithm”.

FIG. 3 shows a system (301) for TRAST experiments. The beam of an excitation laser (302) is modulated by an acousto-optical modulator (303, AOM), reflected by a dichroic mirror (304, DM), and then focused by an objective (305) onto a fluorescent sample (306). The fluorescence then generated in the sample is collected by the same objective (305), passed through the DM (304) and an emission filter (307, EF), and then directed by an objective (308) to a detector/camera (309), which registers an average fluorescence intensity, <F>, for the particular excitation modulation applied.

In TRAST experiments, the modulation of the excitation intensity can in principle be systematically varied in several ways. Typically, square-wave excitation pulse trains are applied (310), with low duty cycles (1:100 or lower) to avoid dark state population pile-up effects, and with the time-averaged fluorescence, <F>, monitored as a function of the duration, w, of the rectangular excitation pulses.

By using the values of the fluorescence, as detected by the detector (309), a so-called TRAST curve (311) may be generated, where <F>, normalized to 1 for short w, is plotted versus w. By such TRAST curves, the population and kinetics of dark photo-induced states, such as T₁, \dot{R}⁻ and \dot{R}⁻ states, can be determined, without the need of time-resolution on the detection side, and in a widely applicable manner. Notably, from a recorded TRAST curve we see that for long w (e.g. wₙ, 312, in the figure)<F> has decreased, indicating that a large fraction of the fluorophores has entered into a dark state. For shorter w, on the other hand (e.g. w₁, 313, in the figure), almost no dark state population build-up is seen.

From FIG. 3 it follows that by applying (in this case rectangular) excitation pulse trains, and by varying a parameter (in this case the duration, w) of the pulses so that dark transient states are populated to different extents, it is possible to vary the degree to which such states are generated.

Due to the pulse-train modulation on the excitation light, the fluorescence intensity is periodical. Thus, the TRAST technique, on the detection side, is intrinsically compatible with the phase-sensitive detection approach (performed by a hardware or a digital lock-in amplifier) if the time-resolved fluorescence intensity is measured by a detector with sufficiently high time resolution. An additional periodic modulation of the excitation light can also be added onto the pulse-train modulation, as a basis for phase-sensitive detection. Use of phase sensitive detection, or lock-in detection (as described e.g. in M L Meade 1982 J. Phys. E: Sci. Instrum. 15 395), can filter out a weak signal from a huge background, with a typical increase in the signal-to-noise ratio by a few orders of magnitude. Use of lock-in detection together with the TRAST approach can further release the requirements of the excitation light intensity applied to allow orders of magnitude smaller PDT precursor state populations and their kinetics to be detected. Thus, the present disclosure may comprise a means for phase-sensitive detection, for example as disclosed above.

For increased therapeutic depths in PDT, several PS compounds have been developed with excitation by near-IR (NIR) light, which is less scattering, non-ionizing, causes no damage to DNA, and is thus essentially harmless to normal cells. In addition to such compounds, the presently disclosed system and its related methods are applicable also on NIR compounds, as exemplified by the experimental data of IR700 in FIG. 6C, and with the advantages that come with excitation in this wavelength range.

To reach even more deeply located regions, and as a means to apply excitation light from multiple positions onto a PDT treatment region, excitation light, as described above, can also be delivered to the target (e.g. a tumor) via optical fibers, for example as in interstitial PDT (IPDT), via the fiber ends at a treatment region. FIG. 4 shows a system for IPDT (401) according to an embodiment of the present disclosure. The system comprises optical fibers (402) coupled to a fiber switch (403) for guiding excitation light from one or several lasers (404) to the region of PDT treatment (405).

The excitation light can be measured by these optical fibers (402). Light transmission signals between these optical fibers can be utilized to assess the treatment-induced variation in the effective attenuation coefficient of the tissue to provide real-time treatment feedback based on light dose threshold models for PDT. Emitted fluorescence light from the PS at the treatment region (405) can also be collected by the fibers, using different source and detector fiber pairs, as described elsewhere herein. By detecting the light, by a detector (406), a processing unit (not shown) can be used to reconstruct the spatial PS distribution within and around the PDT treatment location. Typically, the system (401) comprises a control unit arranged to communicate with, e.g. to control, other parts of the system, such as the light source (e.g. lasers), the fiber switch(es), and/or the detectors.

From e.g. Fluorescence Correlation Spectroscopy (FCS) studies on organic fluorophores, Journal of Physical Chemistry A. 2007; 111(3):429-40, “Strategies to improve photostabilities in ultrasensitive fluorescence spectroscopy” and Anal Chem. 1998; 70(13):2651-9, “Photobleaching of fluorescent dyes under conditions used for single-molecule detection: Evidence of two-step photolysis”, it has been found that excitation to higher excited singlet states (higher states than S₁) can lead to significantly higher yields of triplet state or \dot{R}⁺/\dot{R}⁻ state formation. Likewise, excitation to higher triplet states (higher states than T₁) can promote formation of \dot{R}⁺ and \dot{R}⁻ states. Modulation of the excitation light so that higher excited singlet and/or triplet states in the PS compounds is promoted (for instance by applying shorter pulses effectuating higher Φ_{exc}) is therefore also included as a possible modulation strategy of the presently disclosed system and the related methods.

In one embodiment of the present disclosure, the system is configured for use in a PDT treatment and/or a medical treatment, such as a cancer treatment.

In one embodiment of the present disclosure, the system is arranged to perform a pulse-train modulation of the excitation light, typically the monitoring excitation light. This may for example be carried out by modulating one or more of the following parameters of the excitation light, typically the monitoring excitation light: pulse duration, duration between pulses, pulse shape in space and time, polarization, wavelength and/or excitation intensity within the pulses, typically where said pulse-train consists of one or more pulses.

The excitation is typically performed by one or several lasers, alternatively LEDs, where modulation can be performed by modulating the output beam of the laser(s)/LED(s) via the laser output control directly, or by modulating the output beam(s) by use of one or several optical modulators, e.g. acousto-optic or electro-optic modulators (AOMs or EOMs). Modulated excitation can thus be generated in a similar way, as practiced for TRAST imaging/spectroscopy.

In one embodiment of the present disclosure, the excitation light source is arranged to illuminate the treatment location comprising the PS, and wherein the PS comprises, or consists of, one or more fluorescent organic molecules, typically consisting of one excitable and one corresponding emissive state, and one or several non-emissive states, excitable or not; and/or one or more nanoparticles having corresponding properties, e.g. consisting of one excitable and one corresponding emissive state, and one or several non-emissive states, excitable or not.

In addition to organic molecules as PS compounds, typically consisting of one excitable and one corresponding emissive state, and one or several non-emissive states, excitable or not, there are also PS compounds, which typically have multiple excitable and emissive states, and with different kinetics of these states. An example of a fluorophore with two such states is N-nonyl Acridine Orange (NAO), which states also can be differently populated upon modulated excitation. With several excitable and emissive states of a PS comes the possibility to add an additional excitation light source, at the same or at a different wavelength, with a pulse-train modulation superimposed on the modulation of the original excitation light. Thereby, additional modulation parameters of the excitation light can be added, for further manipulation and control of PDT precursor state populations in the PS, and with that, additional possibilities for local enhancement of PDT precursor state populations at a PDT treatment location.

In addition to PS compounds composed of one fluorophore, also other PS compounds for PDT, having several fluorophores within one entity, such as antibodies, or nanoparticles or vesicles associated with several absorbing and emitting species, are suitable PS compounds, and where also additional advantages can be obtained when they are used with the presently disclosed embodiment.

Moreover, PS compounds can comprise, or be associated with, a nanoparticle or vesicle, loaded with a drug, where the population of photo-induced states in one or several PS associated with the same nanoparticle or vesicle can trigger the release of the drug from the nanoparticle or vesicle, also referred to as uncaging, and used with IR700 as a PS. In such systems, other photo-induced states than the more typical PDT precursor states of T₁, \dot{R}⁺ and \dot{R}⁻ may trigger the drug release, such as photo-isomerized states within the PS compounds associated with a nanoparticle or vesicle.

In one embodiment of the present disclosure, the system is arranged to obtain the PDT precursor state data by measuring the average fluorescence intensity of the PS, typically during at least one wavelength interval and/or during at least one excitation pulse-train modulation.

In one embodiment of the present disclosure, the system is arranged to obtain the PDT precursor state data by measuring and/or deriving a time-resolved fluorescence intensity of the PS during at least one wavelength interval and/or during at least one excitation pulse-train modulation.

In one embodiment of the present disclosure, the system comprises means for phase-sensitive detection, for example as disclosed above.

In one embodiment of the present disclosure, the system is arranged to obtain the PDT precursor state data by measuring and/or deriving the signal amplitude of a time-resolved fluorescence intensity of the PS using a lock-in detection approach during at least one excitation pulse-train modulation.

In one embodiment of the present disclosure, the system is arranged to perform excitation light-induced modification of the effective rates of PDT precursor state formation, such as by being arranged to provide an excitation light that results in an excitation to higher triplet and/or singlet states of the PS.

In one embodiment of the present disclosure, the system comprises an optical fiber arrangement between, at least partly, the light source and the one or more treatment locations, and wherein the optical fiber arrangement is configured for guiding the pulse-train modulated excitation light and for locally collecting fluorescence of the PS.

The optical fiber arrangement may comprise one or more first optical fibers between, at least partly, the light source and the one or more treatment locations. The one or more first optical fibers may preferably be configured for guiding the excitation light, such as the monitoring excitation light and/or the therapeutic excitation light. Preferably, the optical fiber arrangement is configured to also collect fluorescence, preferably of the PS at and around the treatment location.

The presently disclosed system, and the related methods, may be arranged such that collecting fluorescence may be done by having the one or more first optical fibers arranged to locally collect fluorescence of the PS at said treatment location(s) and the surrounding region(s). Alternatively or additionally, the fluorescence may be collected by having one or more second optical fibers arranged to locally collect fluorescence of the PS at one or more secondary location(s), said secondary location(s) being different from the locations at which the first optical fibers are arranged to collect fluorescence from.

In one embodiment of the present disclosure, the system is arranged to identify, based on the PDT precursor state data, microenvironmental differences between the treatment location and the surrounding regions that affect one or more PDT precursor states of the PS, and wherein the system is further arranged to adapt the therapeutic excitation light such that said excitation light selectively enhances a population of one or more PDT precursor states in a PS at the treatment location(s). In this way toxic effects of the PDT at the intended treatment location can be enhanced, without correspondingly enhancing unwanted side-effects in locations surrounding the intended treatment location.

In one embodiment of the present disclosure, the monitoring excitation light is a pulsed light, and the therapeutic excitation light is a pulsed and/or continuous-wave light.

In one embodiment of the present disclosure, the system is arranged to excite the treatment location comprising a PDT adjuvant, such as a compound configured to promote formation of PDT precursor states in the PS, by enhancement of intersystem crossing, photo-ionization or photo-radical state formation.

In one embodiment of the present disclosure, the system is arranged to excite, by the excitation light, a PDT adjuvant, wherein the PDT adjuvant is configured to locally absorb said excitation light, such as the monitoring excitation light and/or the therapeutic excitation light, and to transfer parts of the absorbed energy to activate the PS, or wherein the PDT adjuvant is a heavy-atom, spin-label, electron-donating or electron-accepting compound, which at proximity with the PS, including being bound to the same or different targeting molecule, binding to the same or different, but closely located targets of the treatment location, or following collisional encounters with the PS intra-cellularly, extra-cellularly, or within membranes, enhances e.g. the intersystem crossing and/or photo-ionization rates of the PS.

The system may for example comprise a PDT adjuvant delivery arrangement for delivery of the PDT adjuvant to the one or more treatment locations, such as wherein the PDT adjuvant delivery arrangement comprises or consists of a hollow section of an optical fiber used for excitation and/or fluorescence detection, or wherein the PDT adjuvant delivery arrangement comprises or consists of a separate channel, typically in the vicinity of an optical fiber used for excitation and/or fluorescence detection.

In one embodiment of the present disclosure, the sensing unit is arranged to allow for detection of fluorescence of multiple regions, or of fluorescence fed in parallel via multiple optical fibers.

In one embodiment of the present disclosure, the sensing unit is a point detector and/or a multi-pixel camera.

In general, treatment selectivity typically stems from two major effects:

First, the environment at a treatment location, e.g. the TME of a tumor, is often different than in surrounding tissue, with respect to oxygenation, redox conditions etc. These environmental conditions can strongly affect the PDT precursor states, such that a certain excitation can generate different PDT precursor state populations in a tumor, compared to around the same tumor. Knowledge about the different environmental conditions, and their effect on the PDT precursor state generation, allows for the possibility to adapt the therapeutic excitation light to maximize the contrast in this generation between the treatment location and its surroundings.

Second, the effects of adjuvants are also typically environment dependent, which can give selectivity in the same way as for the adaptation of the excitation light. Moreover, by the application of the adjuvants with a spatial selectivity, PDT precursor state enhancements can be further confined to the treatment location.

The presently disclosed system, and as well as the related methods and computer program products are typically arranged to perform, and/or to be included in, at least a part of a PDT treatment of a patient, such as a cancer treatment.

Typically, the excitation light applied at the treatment region is modulated in the time range of the PDT precursor state dynamics of the PS. From the fluorescence response of the PS compounds to this excitation, and from how this response depends on the excitation modulation, or between modulated and CW excitation, the PDT precursor states can be monitored, within and around the location of the PDT. This monitoring of the PDT precursor states and the subsequent adaptation of the excitation light and/or the provision of PDT adjuvants to the treatment location, can be done in parallel, iteratively, before, during and/or after PDT treatment to optimize the PDT effects.

In a further aspect, the present disclosure relates to a method, for example a method included in a PDT treatment or a method for PDT treatment. The presently disclosed embodiment typically comprises one or more excitation light sources that are arranged to illuminate a treatment location with excitation light. The excitation light source is preferably arranged to independently provide monitoring excitation light and therapeutic excitation light. Typically the monitoring excitation light is pulse-train modulated, and the therapeutic excitation light is pulse-train modulated and/or provided as a continuous wave.

The method is typically arranged to comprise the use of a PS that is arranged to absorb said excitation light and thereby can undergo transitions into PDT precursor states, such as triplet (T₁), photo-reduced (\dot{R}⁻) and photo-oxidized (\dot{R}⁺) states. Typically, wherein in correlation to which states, toxic effects can be generated, such as formation of free radicals, reactive oxygen species (ROS), and singlet oxygen, or cell membrane damage, or other cytotoxic effects.

Advantageously, the method comprises one or more iterative steps of:
performing a pulse-train modulation of the monitoring excitation light characteristics in the time range of the PDT precursor state transitions of the PS,
obtaining PDT precursor state data by monitoring the fluorescence emitted by the PS, said PDT precursor state data indicative of the PDT precursor state kinetics and population of the PS,
adapting the therapeutic excitation light (CW or pulsed) and/or the possible additional provision of a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

It should be noted that the step of obtaining may be performed one or more times together with the step of performing a pulse-train modulation, for example at least one time. Additionally, the step of adapting may be performed before, in-parallel or after the steps of performing and obtaining. Thus the respective order of the steps is not necessarily important.

Thus, the build-up of PDT precursor states in a PS, subject to a certain excitation modulation (the step of performing a pulse-train modulation), can be quite different at the location of a lesion to be treated, compared to at a location close to this lesion, or at the location of the lesion over time.

Monitoring the PDT precursor state population(s) of a PS at and around the location of a lesion, as part of a PDT treatment procedure (the step of obtaining), typically has two major purposes: First, it can add spatial selectivity to the PDT treatment. Based on feedback from this monitoring, the treatment excitation (the step of adapting) can be adapted to optimize PDT precursor state populations of the PS at the location of the lesion and minimize the same populations in surrounding regions, where the local environmental conditions (and thus likely also the PDT precursor state kinetics) are different than at the lesion. Second, it makes it possible to iteratively monitor the PDT precursor state build-up over time (by the steps of obtaining and of performing a pulse-train modulation) and adapt the excitation light (the step of adapting) to for example maintain high populations of the precursor state(s) at the lesion, during the course of the PDT treatment, thereby enhancing the effect of this treatment. Third, it provides feedback on the effects of possibly added PDT adjuvant compounds, on the PDT precursor state populations and kinetics, at and around the treatment location. Thereby, the spatial and temporal supply of PDT adjuvants can be likewise adapted for enhancing the treatment effect(s).

In one embodiment of the present disclosure, the step of adapting the pulse-train modulation comprises or consists of modulating the excitation light by one or more of the following parameters: pulse duration, duration between pulses, pulse shape in space and time, polarization, wavelength and/or excitation intensity within the pulses, typically where said pulse-train consists of one or more pulses.

In one embodiment of the present disclosure, the PS comprises, or consists of, one or more fluorescent organic molecules and/or nanoparticles.

In one embodiment of the present disclosure, the PS comprises, or consists of, a molecule with high affinity to a target structure at the treatment location.

In one embodiment of the present disclosure, the PS is associated with, or comprises, a therapeutic compound, and wherein at least one PDT precursor state of the PS promotes the dissociation of said therapeutic compound.

In one embodiment of the present disclosure, the PS comprises a carrier unit, such as a nanoparticle or a small vesicle, to which the therapeutic compound is associated before being disassociated from said carrier unit, in response to the PS being excited into one of said PDT precursor states that promotes dissociation of said compound.

In one embodiment of the present disclosure, the PDT precursor state data is obtained by measuring an average fluorescence intensity of the PS during at least one wavelength interval, and during at least one excitation pulse-train modulation.

In one embodiment of the present disclosure, the PDT precursor state data is obtained by measuring a time-resolved fluorescence intensity of the PS during at least one wavelength interval, and during at least one excitation pulse-train modulation.

In one embodiment of the present disclosure, the system comprises means for phase-sensitive detection, for example as disclosed elsewhere herein.

In one embodiment of the present disclosure, the PDT precursor state data is obtained by measuring and/or deriving the signal amplitude of a time-resolved fluorescence intensity of the PS using a lock-in detection approach during at least one excitation pulse-train modulation.

In one embodiment of the present disclosure, the step of adapting the pulse-train modulation of the excitation light, comprises excitation light-induced modification of the effective rates of PDT precursor state formation, such as by modulating the excitation light in order to induce higher excited triplet and/or singlet states of the excited PS.

In one embodiment of the present disclosure, the method comprises a step of providing the pulse-train modulated excitation light to the treatment location by an optical fiber arrangement between, at least partly, the light source and the one or more treatment locations, and wherein the optical fiber arrangement is configured for guiding the pulse-train modulated excitation light and for locally collecting fluorescence of the PS at the treatment location.

In one embodiment of the present disclosure, the optical fiber arrangement comprises one or more first optical fibers between, at least partly, the light source and the one or more treatment locations for guiding the pulse-train modulated excitation light, and wherein the optical fiber arrangement further comprises one or more second optical fibers arranged to locally collect fluorescence of the PS at one or more secondary location(s), said secondary location(s) being different from the treatment location(s).

In one embodiment of the present disclosure, the step of adapting the pulse-train modulation of the excitation light comprises identifying, based on the precursor state data, microenvironmental differences between the treatment location(s) and the surrounding region(s) that affect one or more PDT precursor states of the PS, and wherein the method is further arranged to adapt the pulse-train modulation of the therapeutic excitation light (CW or pulsed) such that said excitation light selectively enhances a population of one or more PDT precursor states in a PS at the treatment location(s).

In one embodiment of the present disclosure, the method comprises a step of providing a PDT adjuvant compound to the treatment location.

A PDT adjuvant, or adjuvant, as used herein refers to a compound that can modify the effects on dark transient state transitions on fluorophores. It has been found that e.g. anti-oxidants, such as ascorbic acid, and fluorescence quenchers known to enhance ISC rates, such as potassium iodide, can have prominent effects on the transient states of a fluorophore, but also with large variations in the effects, depending on not only the local environment, but also on the excitation conditions, and on how a certain excitation light dose is distributed in time over a sample, Journal of Physical Chemistry A. 2007; 111(3):429-40, “Strategies to improve photostabilities in ultrasensitive fluorescence spectroscopy”. In principle, since such compounds can likewise also influence transitions of PDT precursor states in a PS used for PDT, local supply of such compounds (i.e. PDT adjuvants) can offer a means to additionally adjust and optimize PDT effects.

In one embodiment of the present disclosure, the method is arranged to excite, by the excitation light, the PDT adjuvant, wherein the PDT adjuvant is a compound configured to locally absorb said excitation light and to transfer parts of the absorbed energy to activate the PS, or wherein the PDT adjuvant is a heavy-atom, spin-label, electron-donating or electron-accepting compound, which at proximity with the PS, including being bound to the same or different targeting molecule, binding to the same or different, but closely located targets of the treatment location, or following collisional encounters with the PS intra-cellularly, extra-cellularly, or within membranes, enhances the PDT precursor state formation of the PS, e.g. by enhanced intersystem crossing and/or enhanced photo-ionization rates of the PS.

In one embodiment of the present disclosure, the PDT adjuvant is delivered to the one or more treatment locations by a PDT adjuvant delivery arrangement, such as wherein the PDT adjuvant delivery arrangement comprises or consists of a hollow section of an optical fiber used for excitation and/or fluorescence detection, or wherein the PDT adjuvant delivery arrangement comprises or consists of a separate channel, typically in the vicinity of an optical fiber used for excitation and/or fluorescence detection.

In one embodiment of the present disclosure, the PDT adjuvant and the PS are conjugated to two separate binding molecules, which can bind to the same target or different targets of the treatment location.

In one embodiment of the present disclosure, the PDT adjuvant is selected to influence one or more local environmental parameters of the treatment location that affects the PDT precursor state formation, such as wherein said local environmental parameter is selected from the group including oxygenation and redox conditions.

In one embodiment of the present disclosure, the sensing unit is arranged to allow for detection of fluorescence of multiple regions, or of fluorescence fed in parallel via multiple optical fibers.

In one embodiment of the present disclosure, the sensing unit is a point detector and/or a multi-pixel camera.

In a further aspect, the present disclosure relates to a computer-implemented method comprising the method included in a PDT treatment as disclosed elsewhere herein, including any embodiment thereof.

Thus, the present disclosure may relate to a computer-implemented method, wherein at least one excitation light source illuminates a treatment location with excitation light, including pulse-train modulated monitoring excitation light and/or a pulse-train modulated or continuous wave therapeutic excitation light, wherein a PS absorbs said excitation light and thereby can undergo transitions into PDT precursor states, such as triplet (T₁), photo-reduced (\dot{R}⁻) and photo-oxidized (\dot{R}⁺) states, in correlation to the population and kinetics of which states toxic effects can be generated, such as formation of free radicals, reactive oxygen species (ROS), and singlet oxygen, or cell membrane damage, or other cytotoxic effects, and where the procedure further includes at least one iterative step of:
performing a pulse-train modulation of the monitoring excitation light characteristics in the time range of the PDT precursor state transitions of the PS,
obtaining PDT precursor state data by monitoring the fluorescence emitted by the PS, said PDT precursor state data indicative of the PDT precursor state kinetics and population of the PS,
adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

In yet a further aspect, the present disclosure relates to a computer program product comprising instructions which, when the program is executed by a computer, cause the computer to carry out the steps of the method as disclosed elsewhere herein.

Thus the presently disclosed method may relate to a computer program product comprising instructions which, when the program is executed by a computer, cause the computer to carry out a method wherein at least one excitation light source illuminates a treatment location with excitation light, including pulse-train modulated monitoring excitation light and/or a pulse-train modulated or continuous wave therapeutic excitation light, wherein a PS absorbs said excitation light and thereby can undergo transitions into PDT precursor states, such as triplet (T₁), photo-reduced (\dot{R}⁻) and photo-oxidized (\dot{R}⁺) states, in correlation to which states toxic effects can be generated, such as formation of free radicals, reactive oxygen species (ROS), and singlet oxygen, or cell membrane damage, or other cytotoxic effects, and where the procedure further includes at least one iterative step of:
performing a pulse-train modulation of the monitoring excitation light characteristics in the time range of the PDT precursor state transitions of the PS,
obtaining PDT precursor state data by monitoring the fluorescence emitted by the PS, said PDT precursor state data indicative of the PDT precursor state kinetics and population of the PS,
adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

In a further aspect, the present disclosure relates to an apparatus, such as a computer, comprising a memory comprising the computer program product as disclosed elsewhere herein. Thus the present disclosure may relate to an apparatus, such as a computer, comprising a memory comprising the computer program product comprising instructions which, when the program is executed by a computer, cause the computer to carry out a method wherein at least one excitation light source illuminates a treatment location with excitation light, including pulse-train modulated monitoring excitation light and/or a pulse-train modulated or continuous wave therapeutic excitation light, wherein a PS absorbs said excitation light and thereby can undergo transitions into PDT precursor states, such as triplet (T₁), photo-reduced (\dot{R}⁻) and photo-oxidized (\dot{R}⁺) states, in correlation to which states toxic effects can be generated, such as formation of free radicals, reactive oxygen species (ROS), and singlet oxygen, or cell membrane damage, or other cytotoxic effects, and where the procedure further includes at least one iterative step of:
performing a pulse-train modulation of the monitoring excitation light characteristics in the time range of the PDT precursor state transitions of the PS,
obtaining PDT precursor state data by monitoring the fluorescence emitted by the PS, said PDT precursor state data indicative of the PDT precursor state kinetics and population of the PS,
adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

In a further aspect, the present disclosure relates to a composition comprising a PS and a PDT adjuvant for use in medical treatment. Preferably, said medical treatment comprises PDT treatment and/or cancer treatment.

In an embodiment of the present disclosure, the PS is configured for PDT and the PDT adjuvant is arranged to promote PDT precursor state formation.

In an embodiment of the present disclosure, the composition is arranged for being used in the method according to any one of claims 17-34.

In an embodiment of the present disclosure, the PS is selected from the group including luminescent PS compounds, organic fluorophores and luminescent nanoparticles.

In an embodiment of the present disclosure, the PS comprises, or consists of, a molecule with high affinity to a target structure at the treatment location.

In an embodiment of the present disclosure, the PS is associated with a therapeutic compound, and wherein at least one PDT precursor state of the PS promotes the dissociation of said therapeutic compound.

In an embodiment of the present disclosure, the PS comprises a carrier unit, such as a nanoparticle or a small vesicle, to which the therapeutic compound is associated before being disassociated from said carrier unit, in response to the PS being excited into one of said PDT precursor states that promotes dissociation of said compound.

In an embodiment of the present disclosure, the adjuvant is selected from the group including heavy-atom, spin-label, electron-donating, electron-accepting compounds, and compounds changing the local environment, in turn changing the PDT precursor state generation and kinetics.

In an embodiment of the present disclosure, the adjuvant is arranged to optimize a PDT precursor state build-up of the PS.

In an embodiment of the present disclosure, the adjuvant is configured to locally absorb incoming radiation and transfer all or parts of the absorbed energy to activate the PS, or a heavy-atom, spin-label, electron-donating or electron-accepting compound, which at proximity to the PS or following collisional encounters with the PS intra-cellularly, extra-cellularly, or within membranes, enhances the intersystem crossing rates or photo-ionization rates of the PS.

In a further aspect, the present disclosure relates to a PS for use in a method of PDT treatment of cancer, wherein the method is as disclosed elsewhere herein.

Thus the present disclosure may relate to a PS for use in a method of PDT treatment of cancer, wherein at least one excitation light source illuminates a treatment location with excitation light, including pulse-train modulated monitoring excitation light and/or a pulse-train modulated or continuous wave therapeutic excitation light, wherein a PS absorbs said excitation light and thereby can undergo transitions into PDT precursor states, such as triplet (T₁), photo-reduced (\dot{R}⁻) and photo-oxidized (\dot{R}⁺) states, in correlation to which states toxic effects can be generated, such as formation of free radicals, reactive oxygen species (ROS), and singlet oxygen, or cell membrane damage, or other cytotoxic effects, and where the procedure further includes at least one iterative step of:
performing a pulse-train modulation of the monitoring excitation light characteristics in the time range of the PDT precursor state transitions of the PS,
obtaining PDT precursor state data by monitoring the fluorescence emitted by the PS, said PDT precursor state data indicative of the PDT precursor state kinetics and population of the PS,
adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

### Items

1. A system comprising:
   an excitation light source arranged to provide pulse-train modulated monitoring excitation light and a pulse-train modulated, or continuous wave, therapeutic excitation light, and wherein the excitation light source is configured to illuminate a treatment location comprising a photosensitizer and wherein the excitation light source is configured to excite the photosensitizer into PDT precursor states;
   a sensing unit configured to monitor the fluorescence emitted by the excited photosensitizer;
   a processing unit configured for at least one iterative step of:
      performing a pulse-train modulation of the monitoring excitation light characteristics in the time range of the PDT precursor state transitions of the photosensitizer,
      obtaining PDT precursor state data by monitoring the fluorescence emitted by the photosensitizer, said PDT precursor state data indicative of the PDT precursor state population of the photosensitizer and/or its kinetics,
      adapting the therapeutic excitation light based on the PDT precursor state data at and around the treatment location, such that toxic effects of the PDT at the intended treatment location are enhanced, without correspondingly enhancing unwanted side-effects in locations surrounding the intended treatment location.
2. The system according to item 1, wherein the system is configured for use in a PDT treatment and/or a cancer treatment.
3. The system according to any one of the preceding items, wherein the system is arranged to perform a pulse-train modulation of the excitation light, such as by modulating one or more of the following parameters: pulse duration, duration between pulses, pulse shape in space and time, polarization, wavelength and/or excitation intensity within the pulses.
4. The system according to any one of the preceding items, wherein the excitation light source is arranged to illuminate the treatment location comprising the photosensitizer, and wherein the photosensitizer comprises, or consists of, one or more fluorescent organic molecules and/or nanoparticles.
5. The system according to any one of the preceding items, wherein the system is arranged to obtain the PDT precursor state data by measuring the average fluorescence intensity of the photosensitizer during at least one wavelength interval, and during at least one excitation pulse-train modulation.
6. The system according to any one of the preceding items, wherein the system is arranged to obtain the PDT precursor state data by measuring a time-resolved fluorescence intensity of the photosensitizer during at least one wavelength interval, and during at least one excitation pulse-train modulation.
7. The system according to any one of the preceding items, wherein the system is arranged to obtain the PDT precursor state data by measuring and/or deriving the signal amplitude of a time-resolved fluorescence intensity of the PS using a lock-in detection approach during at least one excitation pulse-train modulation.
8. The system according to any one of the preceding items, wherein the system is arranged to perform excitation light-induced modification of the effective rates of PDT precursor state formation, such as by being arranged to provide an excitation light that results in an excitation to higher triplet and/or singlet states.
9. The system according to any one of the preceding items, wherein the system comprises an optical fiber arrangement between, at least partly, the light source and the one or more treatment locations, and wherein the optical fiber arrangement is configured for guiding the pulse-train modulated excitation light and for locally collecting fluorescence of the photosensitizer.
10. The system according to item 9, wherein the optical fiber arrangement comprises one or more first optical fibers between, at least partly, the light source and the one or more treatment locations, and wherein the one or more first optical fibers are configured for guiding the excitation light, such as the monitoring excitation light and/or the therapeutic excitation light, and wherein the optical fiber arrangement is configured to collect fluorescence of the photosensitizer:
i. by having the one or more first optical fibers arranged to locally collect fluorescence of the photosensitizer at said treatment location(s) and the surrounding region(s); and/or
ii. by having one or more second optical fibers arranged to locally collect fluorescence of the photosensitizer at one or more secondary location(s), said secondary location(s) being different from the locations at which the first optical fibers are arranged to collect fluorescence from.
11. The system according to any one of the preceding items, wherein the system is arranged to identify, based on the precursor state data, microenvironmental differences between the treatment location and the surrounding regions that affect one or more PDT precursor states of the photosensitizer, and wherein the system is further arranged to adapt the therapeutic excitation light such that said excitation light selectively enhances a population of one or more PDT precursor states in a photosensitizer at the treatment location(s).
12. The system according to any one of the preceding items, wherein the monitoring excitation light is a pulsed light, and the therapeutic excitation light is a pulsed or continuous wave light.
13. The system according to any one of the preceding items, wherein the system is arranged to excite the treatment location comprising a PDT adjuvant, such as a compound configured to promote formation of PDT precursor states in the photosensitizer, by enhancement of intersystem crossing, photo-ionization or photo-radical state formation.
14. The system according to any one of the preceding items, wherein the system is arranged to excite, by the excitation light, a PDT adjuvant, wherein the PDT adjuvant is configured to locally absorb said excitation light, such as the monitoring excitation light and/or the therapeutic excitation light, and to transfer parts of the absorbed energy to activate the photosensitizer, or wherein the PDT adjuvant is a heavy-atom, spin-label, electron-donating or electron-accepting compound, which at proximity with the photosensitizer, including being bound to the same or different targeting molecule, binding to the same or different, but closely located targets of the treatment location, or following collisional encounters with the photosensitizer intra-cellularly, extra-cellularly, or within membranes, enhances the PDT precursor state formation of the photosensitizer, e.g. by enhanced intersystem crossing and/or enhanced photo-ionization rates of the photosensitizer.
15. The system according to any one of items 13-14, wherein the system comprises a PDT adjuvant delivery arrangement for delivery of the PDT adjuvant to the one or more treatment locations, such as wherein the PDT adjuvant delivery arrangement comprises or consists of a hollow section of an optical fiber used for excitation and/or fluorescence detection, or wherein the PDT adjuvant delivery arrangement comprises or consists of a separate channel, typically in the vicinity of an optical fiber used for excitation and/or fluorescence detection.
16. The system according to any one of the preceding items, wherein the sensing unit is arranged to allow for detection of fluorescence of multiple regions, or of fluorescence fed in parallel via multiple optical fibers.
17. The system according to any one of the preceding items, wherein the sensing unit is a point detector and/or a multi-pixel camera.
18. A method included in a photodynamic therapy (PDT) treatment, wherein at least one excitation light source illuminates a treatment location with excitation light, including pulse-train modulated monitoring excitation light and/or a pulse-train modulated or continuous wave therapeutic excitation light, wherein a photosensitizer absorbs said excitation light and thereby can undergo transitions into PDT precursor states, such as triplet (T₁), photo-reduced (\dot{R}⁻) and photo-oxidized (\dot{R}⁺) states, in correlation to which states toxic effects can be generated, such as formation of free radicals, reactive oxygen species (ROS), and singlet oxygen, or cell membrane damage, or other cytotoxic effects, and where the procedure further includes at least one iterative step of:
   performing a pulse-train modulation of the monitoring excitation light characteristics in the time range of the PDT precursor state transitions of the photosensitizer,
   obtaining PDT precursor state data by monitoring the fluorescence emitted by the photosensitizer, said PDT precursor state data indicative of the PDT precursor state kinetics and population of the photosensitizer,
   adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.
19. The method according to item 18, wherein adapting the pulse-train modulation comprises or consists of modulating the excitation light by one or more of the following parameters: pulse duration, duration between pulses, pulse shape in space and time, polarization, wavelength and/or excitation intensity within the pulses.
20. The method according to any one of items 18-19, wherein the photosensitizer comprises, or consists of, one or more fluorescent organic molecules and/or nanoparticles.
21. The method according to any one of items 18-20, wherein the photosensitizer comprises, or consists of, a molecule with high affinity to a target structure at the treatment location.
22. The method according to any one of items 18-21, wherein the photosensitizer is associated with a therapeutic compound, and wherein at least one PDT precursor state of the photosensitizer promotes the dissociation of said therapeutic compound.
23. The method according to item 22, wherein the PS comprises a carrier unit, such as a nanoparticle or a small vesicle, to which the therapeutic compound is associated before being disassociated from said carrier unit, in response to the photosensitizer being excited into one of said PDT precursor states that promotes dissociation of said compound.
24. The method according to any one of items 18-23, wherein the PDT precursor state data is obtained by measuring an average fluorescence intensity of the photosensitizer during at least one wavelength interval, and during at least one excitation pulse-train modulation.
25. The method according to any one of items 18-24, wherein the PDT precursor state data is obtained by measuring a time-resolved fluorescence intensity of the photosensitizer during at least one wavelength interval, and during at least one excitation pulse-train modulation.
26. The method according to any one of items 18-25, wherein the PDT precursor state data is obtained by measuring and/or deriving the signal amplitude of a time-resolved fluorescence intensity of the PS using a lock-in detection approach during at least one excitation pulse-train modulation.
27. The method according to any one of items 18-25, wherein the step of adapting the pulse-train modulation of the excitation light, comprises excitation light-induced modification of the effective rates of PDT precursor state formation, such as by modulating the excitation light in order to induce higher excited triplet and/or singlet states of the excited photosensitizer.
28. The method according to any one of items 18-27, wherein the method comprises a step of providing the pulse-train modulated excitation light to the treatment location by an optical fiber arrangement between, at least partly, the light source and the one or more treatment locations, and wherein the optical fiber arrangement is configured for guiding the pulse-train modulated excitation light and for locally collecting fluorescence of the photosensitizer at the treatment location.
29. The method according to any one of items 18-27, wherein the optical fiber arrangement comprises one or more first optical fibers between, at least partly, the light source and the one or more treatment locations for guiding the pulse-train modulated excitation light, and wherein the optical fiber arrangement further comprises one or more second optical fibers arranged to locally collect fluorescence of the photosensitizer at one or more secondary location(s), said secondary location(s) being different from the treatment location(s).
30. The method according to any one of items 18-29, wherein the step of adapting the pulse-train modulation of the excitation light comprises identifying, based on the precursor state data, differences between the treatment location and the surrounding microenvironment that affect one or more PDT precursor states of the photosensitizer, and wherein the method is further arranged to adapt the therapeutic excitation light such that said excitation light selectively enhances a population of one or more PDT precursor states in a photosensitizer at the treatment location(s).
31. The method according to any one of items 18-30, comprising a step of providing a PDT adjuvant compound to the treatment location.
32. The method according to item 31, wherein the method is arranged to excite, by the excitation light, the PDT adjuvant, wherein the PDT adjuvant is a compound configured to locally absorb said excitation light and to transfer parts of the absorbed energy to activate the photosensitizer, or wherein the PDT adjuvant is a heavy-atom, spin-label, electron-donating or electron-accepting compound, which at proximity with the photosensitizer enhances the intersystem crossing and/or photo-ionization rates of the photosensitizer.
33. The method according to item 32, wherein a proximity with the photosensitizer includes being bound to the same or different targeting molecule, binding to the same or different, but closely located targets of the treatment location, or following collisional encounters with the photosensitizer intra-cellularly, extra-cellularly, or within membranes.
34. The method according to any one of items 31-33, wherein the PDT adjuvant is delivered to the one or more treatment locations by a PDT adjuvant delivery arrangement, such as wherein the PDT adjuvant delivery arrangement comprises or consists of a hollow section of an optical fiber used for excitation and/or fluorescence detection, or wherein the PDT adjuvant delivery arrangement comprises or consists of a separate channel, typically in the vicinity of an optical fiber used for excitation and/or fluorescence detection.
35. The method according to any one of items 31-34, wherein the PDT adjuvant is selected to influence one or more local environmental parameters of the treatment location that affects the PDT precursor state formation, such as wherein said local environmental parameter is selected from the group including oxygenation and redox conditions.
36. The method according to any one of items 18-35, wherein the sensing unit is arranged to allow for detection of fluorescence of multiple regions, or of fluorescence fed in parallel via multiple optical fibers.
37. The method according to any one of items 18-36, wherein the sensing unit is a point detector and/or a multi-pixel camera.
38. A computer-implemented method comprising the method of any one of items 18-37.
39. A computer program product comprising instructions which, when the program is executed by a computer, cause the computer to carry out the steps of the method of any one of items 18-37.
40. The computer program product of item 39, further comprising instructions to allow dose and excitation light distribution computer programs of PDT or IPDT performing the method steps according to anyone of the items 18-37, and which steps include control of the excitation light modulation characteristics, analyses of the fluorescence intensities upon different excitation light modulation characteristics and determination of corresponding PDT precursor state populations of the photosensitizer compounds locally and globally (tomographically), as well as adaptation of the PDT excitation characteristics to enhance the PDT precursor state.
41. An apparatus, such as a computer, comprising a memory comprising the computer program product of item 39.
42. A composition comprising a photosensitizer and a PDT adjuvant for use in medical treatment.
43. The composition according to item 42, wherein the medical treatment comprises PDT treatment and/or cancer treatment.
44. The composition according to any one of items 42-43, wherein the photosensitizer is configured for PDT and the PDT adjuvant is arranged to promote PDT precursor state formation.
45. The composition according to any one of items 42-44, wherein the composition is arranged for being used in the method according to any one of items 18-37.
46. The composition according to any one of items 42-45, wherein the photosensitizer is selected from the group including luminescent photosensitizer compounds, organic fluorophores and luminescent nanoparticles.
47. The composition according to any one of items 42-46, wherein the photosensitizer comprises, or consists of, a molecule with high affinity to a target structure at the treatment location.
48. The composition according to any one of items 42-47, wherein the photosensitizer is associated with a therapeutic compound, and wherein at least one PDT precursor state of the photosensitizer promotes the dissociation of said therapeutic compound.
49. The composition according to any one of items 42-48, wherein the photosensitizer comprises a carrier unit, such as a nanoparticle or a small vesicle, to which the therapeutic compound is associated before being disassociated from said carrier unit, in response to the photosensitizer being excited into one of said PDT precursor states that promotes dissociation of said compound.
50. The composition according to any one of items 42-49, wherein the adjuvant is selected from the group including heavy-atom, spin-label, electron-donating, electron-accepting compounds, and compounds changing the local environment, in turn changing the PDT precursor state generation and kinetics.
51. The composition according to any one of items 42-50, wherein the adjuvant is arranged to optimize a PDT precursor state build-up of the photosensitizer.
52. The composition according to any one of items 42-51, wherein the adjuvant is configured to locally absorb incoming radiation and transfer parts of the absorbed energy to activate the photosensitizer, or a heavy-atom, spin-label, electron-donating or electron-accepting compound, which at proximity to the photosensitizer or following collisional encounters with the photosensitizer intra-cellularly, extra-cellularly, or within membranes, enhances the intersystem crossing rates or photo-ionization rates of the photosensitizer.
53. A photosensitizer for use in a method of PDT treatment of cancer, wherein the method comprises any one of items 18-37.

1. A system comprising:
an excitation light source arranged to provide pulse-train modulated monitoring excitation light and a pulse-train modulated, or continuous wave, therapeutic excitation light, and wherein the excitation light source is configured to illuminate a treatment location comprising a photosensitizer and wherein the excitation light source is configured to excite the photosensitizer into PDT precursor states;
a sensing unit configured to monitor the fluorescence emitted by the excited photosensitizer;
a processing unit configured for at least one iterative step of:
performing a pulse-train modulation of the monitoring excitation light characteristics in the time range of the PDT precursor state transitions of the photosensitizer,
obtaining PDT precursor state data by monitoring the fluorescence emitted by the photosensitizer, said PDT precursor state data indicative of the PDT precursor state population of the photosensitizer and/or its kinetics,
adapting the therapeutic excitation light based on the PDT precursor state data at and around the treatment location, such that toxic effects of the PDT at the intended treatment location are enhanced, without correspondingly enhancing unwanted side-effects in locations surrounding the intended treatment location.

2. The system according to claim 1, wherein the system is configured for use in a PDT treatment and/or a cancer treatment.

3. The system according to claim 1, wherein the system is arranged to perform a pulse-train modulation of the excitation light, such as by modulating one or more of the following parameters: pulse duration, duration between pulses, pulse shape in space and time, polarization, wavelength and/or excitation intensity within the pulses.

4. The system according to claim 1, wherein the excitation light source is arranged to illuminate the treatment location comprising the photosensitizer, and wherein the photosensitizer comprises, or consists of, one or more fluorescent organic molecules and/or nanoparticles.

5. The system according to claim 1, wherein the system is arranged to obtain the PDT precursor state data by measuring the average fluorescence intensity of the photosensitizer during at least one wavelength interval, and during at least one excitation pulse-train modulation.

6. The system according to claim 1, wherein the system is arranged to obtain the PDT precursor state data by measuring a time-resolved fluorescence intensity of the photosensitizer during at least one wavelength interval, and during at least one excitation pulse-train modulation.

7. The system according to claim 1, wherein the system is arranged to obtain the PDT precursor state data by measuring and/or deriving the signal amplitude of a time-resolved fluorescence intensity of the PS using a lock-in detection approach during at least one excitation pulse-train modulation.

8. The system according to claim 1, wherein the system is arranged to perform excitation light-induced modification of the effective rates of PDT precursor state formation, such as by being arranged to provide an excitation light that results in an excitation to higher triplet and/or singlet states.

9. The system according to claim 1, wherein the system comprises an optical fiber arrangement between, at least partly, the light source and the one or more treatment locations, and wherein the optical fiber arrangement is configured for guiding the pulse-train modulated excitation light and for locally collecting fluorescence of the photosensitizer.

10. The system according to claim Error! Reference source not found., wherein the optical fiber arrangement comprises one or more first optical fibers between, at least partly, the light source and the one or more treatment locations, and wherein the one or more first optical fibers are configured for guiding the excitation light, such as the monitoring excitation light and/or the therapeutic excitation light, and wherein the optical fiber arrangement is configured to collect fluorescence of the photosensitizer:
by having the one or more first optical fibers arranged to locally collect fluorescence of the photosensitizer at said treatment location(s) and the surrounding region(s); and/or
by having one or more second optical fibers arranged to locally collect fluorescence of the photosensitizer at one or more secondary location(s), said secondary location(s) being different from the locations at which the first optical fibers are arranged to collect fluorescence from.

11. The system according to claim 1, wherein the system is arranged to identify, based on the precursor state data, microenvironmental differences between the treatment location and the surrounding regions that affect one or more PDT precursor states of the photosensitizer, and wherein the system is further arranged to adapt the therapeutic excitation light such that said excitation light selectively enhances a population of one or more PDT precursor states in a photosensitizer at the treatment location(s).

12. The system according to claim 1, wherein the monitoring excitation light is a pulsed light, and the therapeutic excitation light is a pulsed or continuous wave light.

13. The system according to claim 1, wherein the system is arranged to excite the treatment location comprising a PDT adjuvant, such as a compound configured to promote formation of PDT precursor states in the photosensitizer, by enhancement of intersystem crossing, photo-ionization or photo-radical state formation.

14. The system according to claim 1, wherein the system is arranged to excite, by the excitation light, a PDT adjuvant, wherein the PDT adjuvant is configured to locally absorb said excitation light, such as the monitoring excitation light and/or the therapeutic excitation light, and to transfer parts of the absorbed energy to activate the photosensitizer, or wherein the PDT adjuvant is a heavy-atom, spin-label, electron-donating or electron-accepting compound, which at proximity with the photosensitizer, including being bound to the same or different targeting molecule, binding to the same or different, but closely located targets of the treatment location, or following collisional encounters with the photosensitizer intra-cellularly, extra-cellularly, or within membranes, enhances the PDT precursor state formation of the photosensitizer, e.g. by enhanced intersystem crossing and/or enhanced photo-ionization rates of the photosensitizer.

15. The system according to claim 13, wherein the system comprises a PDT adjuvant delivery arrangement for delivery of the PDT adjuvant to the one or more treatment locations, such as wherein the PDT adjuvant delivery arrangement comprises or consists of a hollow section of an optical fiber used for excitation and/or fluorescence detection, or wherein the PDT adjuvant delivery arrangement comprises or consists of a separate channel, typically in the vicinity of an optical fiber used for excitation and/or fluorescence detection.

16. The system according to claim 1, wherein the sensing unit is arranged to allow for detection of fluorescence of multiple regions, or of fluorescence fed in parallel via multiple optical fibers.

17. The system according to claim 1, wherein the sensing unit is a point detector and/or a multi-pixel camera.

18. A method included in a photodynamic therapy (PDT) treatment, wherein at least one excitation light source illuminates a treatment location with excitation light, including pulse-train modulated monitoring excitation light and/or a pulse-train modulated or continuous wave therapeutic excitation light, wherein a photosensitizer absorbs said excitation light and thereby can undergo transitions into PDT precursor states, such as triplet (T₁), photo-reduced (\dot{R}⁻) and photo-oxidized (\dot{R}⁺) states, in correlation to which states toxic effects can be generated, such as formation of free radicals, reactive oxygen species (ROS), and singlet oxygen, or cell membrane damage, or other cytotoxic effects, and where the procedure further includes at least one iterative step of:
performing a pulse-train modulation of the monitoring excitation light characteristics in the time range of the PDT precursor state transitions of the photosensitizer,
obtaining PDT precursor state data by monitoring the fluorescence emitted by the photosensitizer, said PDT precursor state data indicative of the PDT precursor state kinetics and population of the photosensitizer,
adapting the therapeutic excitation light and/or providing a PDT adjuvant compound(s) to the treatment location(s), based on the PDT precursor state data, such that toxic effects of the PDT at the intended treatment location are enhanced, without correspondingly enhancing unwanted side-effects in locations surrounding the treatment location.

19-38. (canceled)

39. A computer program product comprising instructions which, when the program is executed by a computer, cause the computer to carry out the steps of the method of claim 18.

40-41. (canceled)

42. A composition comprising a photosensitizer and a PDT adjuvant for use in medical treatment.

43-48. (canceled)

49. The composition according to claim 42, wherein the photosensitizer comprises a carrier unit to which the therapeutic compound is associated before being disassociated from said carrier unit, in response to the photosensitizer being excited into one of said PDT precursor states that promotes dissociation of said compound.

50. The composition according to claim 42, wherein the adjuvant is selected from the group including heavy-atom, spin-label, electron-donating, electron-accepting compounds, and compounds changing the local environment, in turn changing the PDT precursor state generation and kinetics.

51. The composition according to claim 42, wherein the adjuvant is arranged to optimize a PDT precursor state build-up of the photosensitizer.

52. The composition according to claim 42, wherein the adjuvant is configured to locally absorb incoming radiation and transfer parts of the absorbed energy to activate the photosensitizer, or a heavy-atom, spin-label, electron-donating or electron-accepting compound, which at proximity to the photosensitizer or following collisional encounters with the photosensitizer intra-cellularly, extra-cellularly, or within membranes, enhances the intersystem crossing rates or photo-ionization rates of the photosensitizer.

53. (canceled)
